(19) United States
(12) Patent Application Publication
(10) Pub. No.: US 2009/0186318 A1
(43) Pub. Date: Jul. 23, 2009

(54) Laser Surgical Methods

(75) Inventors: Shlomo Assa, Valley Center, CA (US); Steven Jerome Meyer, San Diego, CA (US)

Correspondence Address: FISH & RICHARDSON, PC, P.O. BOX 1022, MINNEAPOLIS, MN, 55440-1022, US

(73) Assignee: INLIGHT CORPORATION, San Diego, CA (US)

(21) Appl. No.: 12/016,851

(22) Filed: Jan. 18, 2008

(51) Int. Cl. A61C 3/00; A61B 18/18

(52) U.S. Cl. 433/215; 606/11

(57) ABSTRACT

Methods, systems, and apparatus for programmable selective ablating or cutting of a targeted area of a material with a laser, producing a succession of pulses of the generated radiation with an energy level, pulse duration, and repetition rate specified to ablate or cut the material without causing harmful side effects; and concentrating the radiation pulses on the targeted material to a spot sufficiently small to cause ablating or cutting of the material; and means to direct the said spot to cover the programmed selected targeted area of a material.

### CROSS-REFERENCE TO RELATED APPLICATIONS

This application is related to U.S. patent application Ser. No. TBD, entitled LASER SURGICAL APPARATUS, to inventors Shlomo Assa, Steve J. Meyer and John Stine, which application was filed on the same day as the present application, and this application is related to U.S. patent application Ser. No. TBD, entitled DISPOSABLE HAND PIECE FOR DENTAL SURGICAL LASER, to inventors Shlomo Assa, Steve J. Meyer, Julie Assa and Gordon J. Foote, which application was filed on the same day as the present application. The disclosures of the above two applications are incorporated herein by reference in their entirety.

### BACKGROUND

This specification relates to laser surgery and to cutting of dental and other hard tissue and non-cellular material.

In dental procedures, it is frequently desirable to remove portions of tooth enamel and dentin, and in certain cases, portions of gum tissue, in an accurately controlled manner and there has been a growing interest in the use of laser radiation for performing such procedures. The use of laser radiation is attractive because, particularly with the aid of optical delivery systems, such radiation can be focused to a very small area and is thus compatible with the dimensional scale of dental procedures. Moreover, laser radiation procedures can be performed without recourse to an anesthetic.

Laser use in dental enamel surgery was reported as early as 1964 using a ruby laser. Although such reports indicated that lasers could be used on dental hard tissue, lasers have not generally been used clinically until recently for surgical processes, including drilling teeth, because of the large amount of damage to nearby tissue that is often associated with such drilling. Pulsed eximer lasers as well as lasers producing infrared radiation have, however, been used recently for soft tissue and bone ablation due to the fact that these types of lasers have been found to do less damage than previous lasers.

The enamel and dentin of a tooth include, as one component, hydroxyapatite, which is in amorphous form in the dentin and crystalline form in the enamel. These portions of a tooth additionally include organic tissues and water, but have no vascular system. Healthy dentin is in mineralized form, while dentin which has experienced decay is in demineralized form. Dentin has a relatively high percentage of organic tissue, around 40 percent, and also a high percentage of water. These percentages increase considerably in decayed dentin.

Tooth pulp and the gum surrounding the teeth consist of vascularized organic tissue containing both hemoglobin and water. Each of these components has a different response to laser radiation. Moreover, it has been found, that hydroxyapatite absorbs laser radiation in the wavelength ranges of 9-11 μm., such as produced by CO₂ lasers, and also in the wavelength range 0.5-1.06 .mu., which includes the wavelength that can be produced by a Nd:YAG laser.

While a particular wavelength may inherently have a cutting effect on enamel or dentin, it has been found that the practical utilization of radiation at such a wavelength for dental procedures is highly dependent on the form in which the radiation is applied, with respect to energy level, pulse duration and repetition rate. Specifically, efforts to apply such radiation in the form of high energy pulses of short duration have been found to produce a highly localized temperature increase, resulting in differential thermal expansion which can cause mechanical damage to the tooth as well as vascular damage to pulp tissue. Conversely, low energy pulses of long duration cause a more widespread heating of the tooth which results in patient discomfort as well as pulp damage due to heating.

The trend today is to use minimally invasive procedure that can repair tooth decay early, while minimizing patient's discomfort. Lasers have proved efficient and precise in other industrial field, promising potentially to better support the current trends.

Another important trend in medical technology in general, and in Dental treatment in particular, is the use of selective area to be treated. The Use of computerized means to distribute laser energy is applied in many cosmetic surgery applications today, and can be utilized similarly for dental treatments.

New detection tools for early detection of tooth decay are spreading fast in the dental sector, including tools and means to generate digital image of dental features, including but not limited to individual teeth or a portion of a tooth, all the way to entire oral cavity.

Assa et al. (U.S. Pat. No. 5,906,609 and U.S. Pat. No. 5,938,657) patented a method and apparatuses to deliver focused laser energy to a selected area. The method and device have means to focus a laser beam and means to move the laser beam in both X and Y direction to be directed to a selected area within the marked outline. These methods are currently used in many different applications in the cosmetic surgery field.

Wolbarsht et al. (U.S. Pat. No. 5,267,856) patented a method of ablating or cutting a selected area of dental hard tissue using Er; YAG laser (at wavelength of 2.94 μm) assistant with water and air mist. Since water will be retained in the microscopic cracks in the hard tissue and since water heavily absorb the particular wavelength it becomes and effective ablating or cutting tool. This method, however, is limited to a free-hand laser focus beam, similarly to all the following methods.

Myers et al. patented a method for removing decay from teeth using a yttrium-aluminum-garnet (YAG) laser for a picosecond to several milliseconds (U.S. Pat. No. 4,818,230). The laser was used to eradicate tooth decay located in the dentin, “without significantly heating the tooth and thus without damage to the nerve”. The disclosure of this patent and all other patents and publications referred to herein is incorporated herein by reference.

A YAG laser has also been used to remove incipient carious lesions and/or stain from teeth (U.S. Pat. No. 4,521,194). This use of a YAG laser was found to slightly fuse the crystals which form the tooth enamel and make the tooth enamel more impervious to decay.

Blum et al. (U.S. Pat. No. 4,784,135) discloses use of an ArF excimer laser as an ultraviolet light source (wavelengths less than 200 nm) to ablatively photodecompose decayed teeth and remove the surrounding enamel.

Erbium is a metallic element of the rare-earth group that occurs with yttrium and is also used as a source of laser irradiation. An Er:YAG laser is a solid-state, pulsed laser which has a maximum emission in the mid-infrared region at 2.94 um. Water absorbs strongly in this region with the water absorption coefficient for radiation produced by an Er:YAG laser being ten times that of radiation produced by a CO₂ laser. Laser surgery performed with an Er:YAG laser apparently results in water in the target tissue absorbing radiant energy and heating to boiling to produce water vapor. The water vapor builds up in pressure at the surgical site until a microexplosion occurs and a small portion of tissue is ablated. A number of publications have discussed the great potential for Er:YAG lasers for tissue, bone and cartilage ablation (e.g., Laryngoscope 100:14, 1990; Lasers in Surgery and Medicine 8:494, 1988; 9:327, 1989; and 9:362, 1990). Radiation from a pulsed Er:YAG laser can be transmitted through optical fibers and its pulse nature allows cooling between pulses

Researchers in Germany have found that pulsed 2.94 um Er:YAG laser radiation in vitro is effective in removal of both dentin and enamel (Hibst and Keller, Lasers in Surgery and Medicine 9:338, 1989). These researchers found that when the duration of the total erbium laser pulse was about 250 microseconds with a pulse train of single spikes of about 1 microsecond each, roughly cone-shaped holes were produced. They also found that with a radiant exposure of 30 J cm⁻², the depth hole in dentin and enamel was proportional to the number of pulses, except at higher numbers of pulses for enamel.

In a companion study, the same researchers used light and scanning electron microscopy to view tooth dentin and enamel exposed to Er:YAG laser radiation (Lasers in Surgery and Medicine 9:345, 1989). Using the same laser treatments as in the companion paper, they found that very few charred or fused zones or cracks were found with the Er:YAG treatment, as compared to CO₂ laser dental surgery. There was also little heating of the tissue surrounding the crater.

Water has been used in conventional dental surgery and in laser dental surgery as a coolant for the tooth after a surgical pulse. For example, the patent of Vassiliadis et al. (U.S. Pat. No. 4,940,411) discloses a dental laser method using a Neodymium:YAG laser. In this invention, water is sprayed on the tooth after a pulse, followed by drying of the tooth prior to a subsequent activation of the pulsed laser. This patent and the work of others stress the importance of keeping the tooth dry during delivery of the laser pulse, especially for any lasers, such as an Er:YAG laser, productive of radiation that is absorbed by water to minimize heating of and damage to the surface of the tooth.

### SUMMARY

This specification describes technologies relating to laser surgery and to cutting of dental and other hard tissue and non-cellular material.

In general, one or more aspects of the subject matter described in this specification can be embodied in one or more methods of performing laser surgery by ablating or cutting a preprogrammed and selected particular area of hard material such as teeth, bones or a none cellular material. The one or more methods can involve the use of (a) a digital image of the selected area to be treated from any of a variety of sources such as, but not limited to, digital camera, digital x-ray, digital image of fluorescence, or a digital reproduced image by means to scan conventionally a paper printed image; (b) means to export the said image to a Personal Computer capable to accept the image, similar to the way digital cameras export digital pictures to a Personal computer; (c) software means to program the selected area and area boundaries based on the digital image imported (e.g., software that is previously installed on the target Personal Computer); (d) means to export the boundaries to the apparatus; (e) means to maneuver a focused radiation within boundaries of a selected area in preprogrammed steps; (f) means to generate pulsed radiation energy with pulse energy and duration that is suitable to ablate or cut the selected hard material such as teeth, bones on other none cellular material; (g) means to direct the said focused radiation to fill the said selected area within the defined boundaries, one layer at a time. Other embodiments of this aspect include corresponding systems, apparatus, and computer program products.

Particular embodiments of the subject matter described in this specification can be implemented to realize one or more of the following advantages. Using the digital image to define and program the selected area by defining the boundaries, and proceeding to guide and direct the focused radiation energy to cover the programmed selected area to ablate or cut the hard material can result in significant advantages, including improved effectiveness for and expanded conditions under which laser surgery can be performed, such as with dental surgery. In some embodiments, the selected area is comprised of preprogrammed and predefined selection of different sizes and shaped areas such as but not limited to circle, square, rectangle, triangle and other shapes. Moreover, using the described techniques, the use of lasers can improve a dentist's productivity by reducing the strain and fatigue associated with treating patients.

The details of one or more embodiments of the subject matter described in this specification are set forth in the accompanying drawings and the description below. Other features, aspects, and advantages of the invention will become apparent from the description, the drawings, and the claims.

### BRIEF DESCRIPTION OF THE DRAWINGS

FIG. 1 is a typical digital picture of a human molar tooth with decay pattern. This picture is typically taken by the treating Dentist prior to the treatment using existing dental digital camera.

FIG. 2 is the schematic view of the outlines of the decay as been defined by the Dentist using the software tools that are part of this invention.

FIG. 3 is a schematic view of the selected outlines filled with laser pulses as the programming of the selected area is completed.

FIG. 4 is the treatment selected preprogrammed area aligned with the tooth decay to explain the importance of the invention.

FIG. 5 is a schematic of a rectangle which is one of the preprogrammed areas described as part of this invention.

FIG. 6 is a schematic of circle which one of the preprogrammed areas described as part of this invention.

FIG. 7 is a description of the step distance between each consecutive radiation focused pulses.

### DETAILED DESCRIPTION

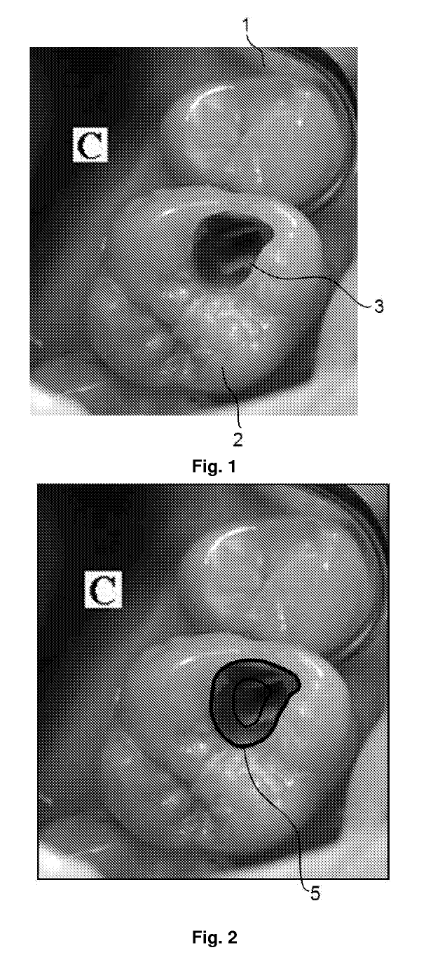

According to the present invention, the method commences with a digital image of tooth decay. FIG. 1 is a typical picture taken by a digital Dental camera. As shown in FIG. 1, human gums 1 support a human molar tooth 2 with tooth decay 3. The photo can be downloaded to a Personal Computer in a conventional way as it is done today. The image can be retrieved from a digital X-Ray of the tooth, or a digital scan of a paper photograph of the tooth.

According to this invention, and using software tools, as described herein, the dentist can use the Personal Computer's pointing device to define the edges of the tooth decay over the displayed digital image, creating one or more boundaries. FIG. 2 shows boundaries 5 overlaid on the digital image from FIG. 1. The boundaries can be scaled by the user entering the image size.

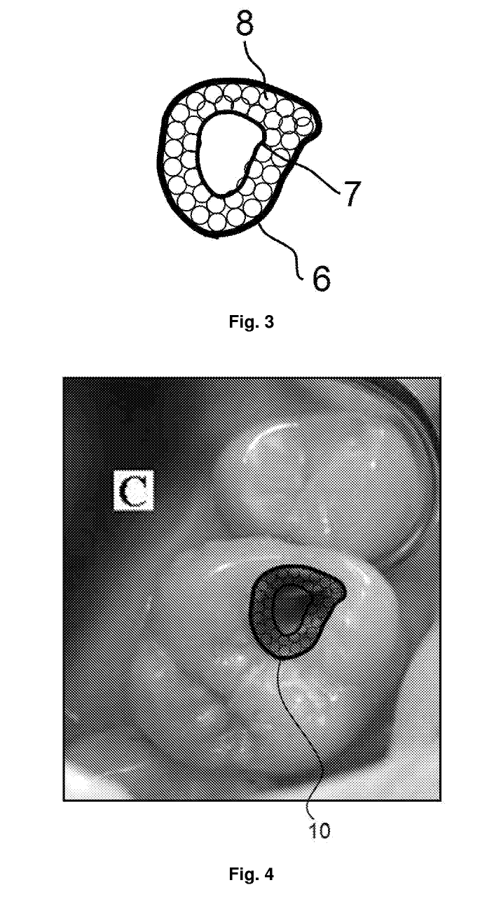

FIG. 3 is the schematic of the selected area defined to be treated having boundaries 6 and 7, by radiation ablation or cutting filled with consecutive focused pulses locations 8. Using the means to move the radiation focused energy spot at distance d of FIG. 7, the apparatus can thus cover the selected area.

The distance d of FIG. 7 is a programmable value of the step size, and it can be varied as a percentage of the radiation focused spot size, such as in a range from 30% to 80%, with the default set at 50%.

FIG. 3 is the defined treatment boundaries of the selected area displayed by the apparatus that is part of this invention. To show the defined treatment boundaries with respect to the tooth, a red diode laser can be used to focus and move a spot around the boundaries in repetition at a rate that is greater than 50 times a second, thereby forming a standing image 10 as shown in FIG. 4. This image at the correct scale can then be aimed and aligned with the tooth decay. The user can then hold the apparatus steady in place and by pressing a command switch, such as a footswitch, the focused radiation spot will be moved from spot to spot with distance d as outlined in FIG. 7 until the last spot is covered. To repeat the process, the user can press the command switch again.

According to the present invention the continuation of laser ablating of the tooth carries will continue with the last selected area boundaries or a scaled down in programmable size, to smaller area until the Dentist will determined that the remain tooth is clean from decay and he will be ready to fill the cavity and finalize the treatment.

The present invention is for a method of performing laser surgery by ablating or cutting a preprogrammed and selected particular area of hard material such as teeth, bones or a none cellular material. In some embodiments, the selected area will be part of a human tooth that has different level of tooth decay that needs to be removed.

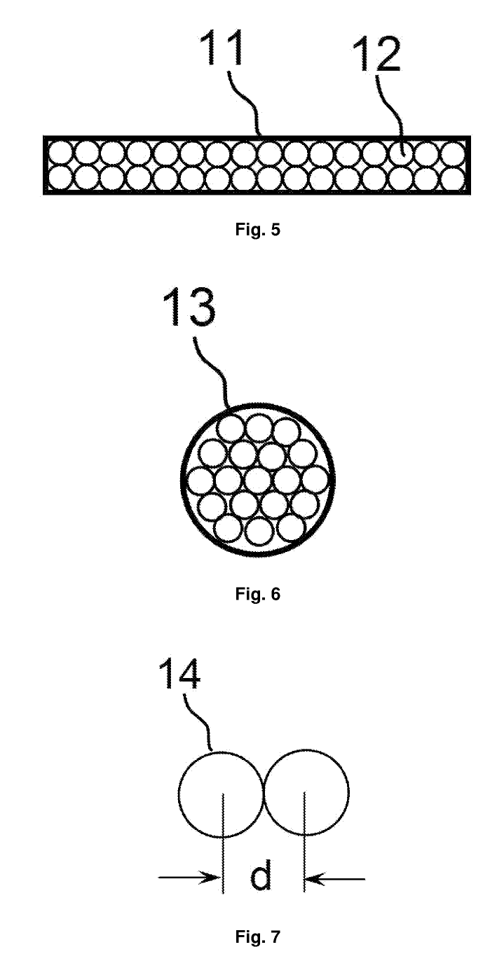

The apparatus that is part of this invention has selection of areas that had been preprogrammed, and according to this invention the shapes can include, but are not limited to, circle, square, rectangle, ellipse, triangle and line, programmed to sizes of 1-4 mm. FIG. 5 is a selected narrow rectangle where the boundary 11 will be displayed by the apparatus using a red laser diode focused beam moving at rate larger than 50 times a second creating a standing image that the user can aim and position over the selected area to treat. The radiation focused pulse locations 12 can then be filled, causing the selected area ablation. FIG. 6 is similarly a circle as a selected area.

The choices of the different preprogrammed shapes and sizes are performed by the user before the ablation process commences. Moreover, the use of preprogrammed shapes and sizes, such as described in connection with FIGS. 5 and 6, need not also use the digital picture since the preprogrammed shapes and sizes can be selected on the fly by the user, and the selected shapes and sizes can be readily seen on the targeted area itself using the visible standing image, as described.

According to this invention, in some embodiments, the radiation source is a CO₂ laser at wavelength of 9.3 μm. The laser emits pulses for duration of 50-75 μseconds, with energy of 5-7 milli-joules per pulse spaced at 2.5 milliseconds apart, or at frequency of 400 Hertz. In addition, some embodiments will have a red diode laser with wavelength of 650 nm. This red visible laser is use to show the physical programmed boundaries.

The laser beam can be focused to a 135 μm by an optical lens with focal distance of 50 mm. The method and apparatus described by this invention includes motorized scanner mirrors capable to direct and maneuver the said focused beams in an area larger than the said pre-selected programmed area.

The apparatus can be used by the operator to select the area with a particular predetermined shape and size. The apparatus can maneuver the focused red diode laser at rate of 50 times per second around the selected boundaries forming a visible standing image of the selected area boundaries that can be used by the operator to aim and align the area with the area on the tooth that need s to be ablated or cut.

According to this invention, the apparatus is provided with an electric activating switch, such as but not limited to, a footswitch, that the operator can use to activate the CO₂ laser to ablate or cut the defined selected area. By activating the said electric switch, the focused laser beam will be directed to the first spot inside the selected area and the X-Y motorized scanner will aim and hold the laser direction at this spot for the duration of about 2.5 milli-second. Within this time the laser will release one pulse.

According to the details of this invention, the system then will move the said focused laser beam distance d mm away from the previous spot along one axis (either X or Y axis) for the said 2.5 milliseconds. FIG. 7 distance d is a programmable value and can be defined as a percentage of the spot size, such as where the percentage is set to 50%, thus d=67.5 μm. Within this period of time the laser will release the next pulse. This process will repeat until the selected area is fully covered by pulses both in X and Y direction. According to the details of this invention, the user can repeat the ablation by using same setting and activating the footswitch again for another layer, or change the setting to different shape or different size, until the Dentist will determine that the selected area is clean from any undesired substance.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a tangible program carrier for execution by, or to control the operation of, data processing apparatus. The tangible program carrier can be a computer-readable medium. The computer-readable medium can be a machine-readable storage device, a machine-readable storage substrate, a memory device, or a combination of one or more of them.

The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

While this specification contains many implementation details, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Thus, particular embodiments of the invention have been described, but other embodiments are within the scope of the following claims. For example, a different method of scanning can be used to form a fixed circular pattern that only varies in size.

What is claimed is:

1. A method of selective ablating or cutting of a targeted area of a material, the method comprising:
receiving a digital image of the targeted area of the material;
defining one or more boundaries of the targeted area;
directing light to form a standing image of the one or more boundaries while the standing image is aimed and aligned with the targeted area of the material; and
directing focused radiation energy to ablate or cut the material with respect to the one or more boundaries that are aimed and aligned with the targeted area.

2. The method of claim 1, wherein the receiving comprises receiving the digital image in a personal computer, and the defining comprises defining the one or more boundaries of the targeted area, including a scale for the one or more boundaries, in response to user input to software tools installed on the personal computer.

3. The method of claim 2, wherein the directing the light and the directing the focused radiation energy comprises producing one or more visually continuous and sized boundaries while ablating or cutting laser energy is directed to the targeted area of the material.

4. The method of claim 2, wherein the defining and the directing the light comprise scaling the one or more boundaries by a predetermined factor of the one or more boundaries.

5. The method of claim 4, wherein a shape of the one or more boundaries is selected from a group consisting of polygons, circles, ellipses, line, rectangle and triangle.

6. The method of claim 4, wherein a shape of the one or more boundaries is selected from a group consisting of square, diamond, rectangle, triangle, pentagon, hexagon, heptagon, and octagon.

7. The method of claim 2, wherein the defining comprises receiving the user input to draw an outline around the targeted area to be treated with energy.

8. The method of claim 2, wherein the directing the focused radiation energy comprises delivering laser pulses with a step distance that is programmed.

9. The method of claim 2, wherein the standing image of the one or more boundaries has a size between 0.1 square millimeters and 16 square millimeters, and wherein the directing the light comprises forming the standing image using light from a laser.

10. The method of claim 2, wherein the energy is from a CO₂ laser at 9.3 μm or 9.6 μm or 10.6 μm.

11. The method of claim 2, wherein the energy is from a Nd;YAG laser at 1.06 μm or 1.32 μm or 0.532 μm.

12. The method of claim 2, wherein the energy is from Er; YAG laser at 2.94 μm or Er:YSGG laser at 2.78 μm.

13. The method of claim 2, wherein the energy is produced by a coherent light source.

14. The method of claim 2, wherein the energy is focused on the surface in a spot having a diameter less than 1.0 millimeter.

15. The method of claim 2, wherein the energy is focused on the material in a spot having a diameter between 150 μm and 250 μm.

16. The method of claim 2, wherein the directing the focused radiation energy comprises delivering the energy continuously over substantially all of the one or more boundaries.

17. The method of claim 2, wherein the directing the focused radiation energy comprises delivering the energy intermittently over the one or more boundaries.

18. The method of claim 2, wherein the directing the focused radiation energy comprises delivering the energy in a predetermined pattern over the one or more boundaries.

19. The method of claim 18, wherein the predetermined pattern is adapted for performing dental cavity preparation.

20. The method of claim 2, wherein the energy ablates or cuts a surface layer of carries on a human live tooth.
